(19) United States
(12) Patent Application Publication
(10) Pub. No.: US 2026/0297286 A1
(43) Pub. Date: Oct. 1, 2026

(54) METHOD FOR DEPOLYMERISING A POLYESTER COMPRISING POLYETHYLENE TEREPHTHALATE BY RECIRCULATING AN OLIGOMER EFFLUENT

(71) Applicants: IFP ENERGIES NOUVELLES, Rueil-Malmaison (FR); JEPLAN, INC., Kawasaki City (JP)

(72) Inventors: Adrien MEKKI-BERRADA, Rueil-Malmaison Cedex (FR); Nicolas VIN; Frederic FAVRE; Damien LEINEKUGEL LE COCQ, Rueil-Malmaison Cedex (FR); Olivier THINON, Rueil-Malmaison Cedex (FR)

(73) Assignees: IFP ENERGIES NOUVELLES, Rueil-Malmaison (FR); JEPLAN, INC., Kawasaki City (JP)

(21) Appl. No.: 19/478,908

(22) PCT Filed: May 6, 2024
(86) PCT No.: PCT/EP2024/062390

§ 371 Date: Oct. 27, 2025

(30) Foreign Application Priority Data
May 12, 2023 (FR) FR2304723

(51) Int. Cl. C08J 11/24; C07C 29/09; C07C 51/09

(52) U.S. Cl. CPC C08J 11/24; C07C 29/095; C07C 51/09; C08J 2367/02

(57) ABSTRACT

The invention relates to a process for depolymerizing a polyester feedstock comprising PET. The process comprises a step a) of conditioning the polyester feedstock, a depolymerization step b) which implements several reaction sections (A) (B) (N), a step of separation (c) of the diol, a step of separation (d) of a heavy impurities effluent, optionally a decolourization step (f) followed by a crystallization step (g). The heavy impurities effluent undergoes a separation step (e) to produce two fractions: a first fraction, at least 70% by weight of which is sent to the second reactor (B), and a second fraction which is at least partially discharged from the process.

### TECHNICAL FIELD

The invention relates to a process for depolymerizing a polyester, in particular terephthalate polyester, comprising polyethylene terephthalate (PET), for the purpose of recycling it. More particularly, the invention relates to a process for depolymerizing a polyester feedstock comprising PET, to produce a diester monomer, said process having an optimized recycling of an oligomers effluent.

### PRIOR ART

The chemical recycling of polyester, in particular of polyethylene terephthalate (PET), has been the subject of numerous studies aimed at decomposing the polyester recovered in the form of waste into monomers which could be used again as feedstock for a polymerization process.

Numerous polyesters result from networks for collecting and sorting materials. In particular, polyester, in particular PET, may originate from the collection of bottles, trays, films, resins and/or fibres composed of polyester (for instance textile fibres, tyre fibres). The polyester resulting from collecting and sorting channels is referred to as polyester to be recycled.

PET to be recycled can be classified into four main categories:
clear PET, predominantly consisting of transparent colourless PET (in general at least 60% by weight) and of transparent azure PET, which does not contain pigments and may be employed in mechanical recycling processes,
dark, or coloured, PET (green, red, etc.), which can generally contain up to 0.1% by weight of dyes or pigments but remains transparent, or translucent;
opaque PET, which contains a significant amount of pigments at contents typically varying between 0.25 and 5.0% by weight to opacify the polymer. Opaque PET is increasingly being used, for example, in the manufacture of food containers, such as milk bottles, in the composition of cosmetic, plant-protection or dye bottles;
multilayer PET, which consists of layers of polymers other than PET or a layer of recycled PET between layers of virgin PET (i.e. PET that has not undergone recycling), or an aluminium film for example. Multilayer PET is used after thermoforming to make packaging such as trays.

Collection channels, which supply recycling channels, are structured differently depending on the country. They are evolving to maximize the amount of plastic recovered in waste depending on the nature and quantity of streams and sorting technologies. The recycling channel for these streams generally consists of a first stage of conditioning in the form of flakes, during which bales of raw packaging are unpackaged and the containers washed, sorted and crushed and then further purified and sorted to produce a stream of flakes containing generally less than 1% by mass of “macroscopic” impurities (glass, metals, other plastics, wood, paper card, mineral elements).

The flakes of clear PET can then undergo an extrusion-filtration step to produce extrudates, which are then reusable as a mixture with virgin PET to make new products (bottles, fibres, films). A solid state vacuum polymerization step (known by the acronym SSP) is necessary for food uses. This type of recycling is known as mechanical recycling.

Dark (or coloured) PET flakes can also be recycled mechanically. However, the colouration of the extrudates formed from the coloured streams limits the uses: dark PET is generally used to produce packaging straps or fibres. The outlets are thus more limited in comparison with those of clear PET.

The presence of opaque PET containing pigments at high contents in the PET to be recycled causes problems for recyclers because opaque PET impairs the mechanical properties of recycled PET. Opaque PET is currently collected with coloured PET and is found in the coloured PET stream. In view of the development of the uses for opaque PET, the contents of opaque PET in the stream of coloured PET to be recycled are currently between 5-20% by weight and are tending to increase further. In a few years' time, it will be possible to achieve contents of opaque PET in the coloured PET stream of greater than 20-30% by weight.

However, it has been shown that above 10-15% opaque PET in coloured PET streams, the mechanical properties of recycled PET are impaired (cf. “Impact of the development of opaque white PET on the recycling of PET packaging”, preliminary note of COTREP of May 12, 2013) and prevent recycling in the form of fibres, the main outlet of the channel for coloured PET.

The dyes are natural or synthetic substances, soluble in particular in the polyester material and used to colour the material into which they are introduced. The dyes generally used are of different kinds and often contain heteroatoms of O and N type, and conjugated unsaturations, such as, for example, quinone, methine or azo functions, or molecules such as pyrazolone and quinophthalone. Pigments are finely divided substances which are insoluble, in particular in the polyester material, and which are used to colour and/or opacify the material into which they are introduced. The main pigments used to colour and/or opacify polyesters, in particular PET, are metal oxides such as TiO₂, CoAl₂O₄, Fe₂O₃, silicates, polysulfides, and carbon black. The pigments are particles with a size generally of between 0.1 and 10 μm and predominantly between 0.4 and 0.8 μm. The complete removal of these pigments by filtration, which is necessary in order to envisage recycling the opaque PET, is technically difficult as they have an extremely high clogging capability.

The mechanical recycling of coloured and opaque PET is therefore extremely elaborate. Document FR 3053691 A1 describes a process for depolymerizing a polyester feedstock comprising opaque PET and in particular 0.1 to 10% by weight of pigments, by glycolysis in the presence of ethylene glycol. An effluent of purified bis(2-hydroxyethyl) terephthalate (BHET) is obtained after particular separation and purification steps. This document envisages the possibility of a reactive extrusion in a first stage of conditioning the feedstock in order to initiate the depolymerization reaction. It also mentions the recycling of the heavy residues separated during the purification steps, to be treated with the polyester feedstock.

Document FR 3105236 describes an improvement of the process for depolymerization by glycolysis of a polyester feedstock comprising PET, and in particular that of document FR 3053691. The improvement consists in particular in optimizing the phase of conditioning the polyester feedstock by mixing the feedstock with at least one recycled oligomer residue effluent in the presence of diol, upstream of its introduction into the depolymerization step.

The present application proposes to increase the yield of the depolymerization process by optimizing the use of the heavy impurities effluent obtained during the separation of the effluent rich in liquid monomers.

### SUMMARY OF THE INVENTION

The present invention relates to a process for depolymerizing a polyester feedstock comprising PET, said process comprising:
a) a conditioning step, fed at least by said polyester feedstock, to produce a conditioned feedstock stream;
b) a depolymerization step implementing a first reaction section and at least one second reaction section, said at least one second reaction section operating at a temperature strictly lower than the temperature of said first reaction section, the first reaction section being fed at least by the conditioned feedstock stream and optionally by a first diol makeup, said at least one second reaction section being fed by the effluent of the first reaction section, and by a recycled oligomers effluent and optionally by a second diol makeup, so that the total amount of diol feeding said step b) is adjusted to 1 to 20 moles of diol per mole of diester feeding said step b), said step b) being operated at a temperature of between 18° and 300° C. and with a residence time of between 0.334 and 10 h;
c) a step for separating the diol, fed at least by the effluent of step b), operated at a temperature of between 100 and 250° C., at a pressure lower than that of step b) and producing a diol effluent and a liquid monomer-rich effluent;
d) a step for separating the liquid monomer-rich effluent from step c) into a heavy impurities effluent and a pre-purified monomers effluent, operated at a temperature of less than or equal to 250° C. and a pressure of less than or equal to 0.001 MPa with a liquid residence time of less than or equal to 10 min,
e) a step for separating said heavy impurities effluent into two fractions: a first fraction of which at least 70% by weight compose the recycled oligomers effluent which feeds step b) and a second fraction which is at least partially discharged from the process;
f) optionally, a step for decolourizing the pre-purified monomers effluent, operated at a temperature of between 10° and 250° C. and at a pressure of between 0.1 and 1.0 MPa in the presence of an adsorbent, and producing a purified monomers effluent,
g) optionally, a step for crystallizing the purified monomers effluent, employing at least one solid production section, operated at a temperature between 0 and 100° C. and at a pressure of between 0.00001 and 1.00 MPa, followed by a solid-liquid separation section, producing a decolourized and purified monomers effluent.

Recycling the oligomers obtained in step e) into the second reaction section of the depolymerization step b) which operates at a lower temperature level than the first reaction section and at a lower temperature than that employed in conditioning step a) has several advantages, in particular:
an improvement in the quality of the produced monomer, in particular a reduction in the proportion of comonomer, especially of BHETdeg, in the monomer product, which is responsible for the greatest loss of yield after oligomers. Indeed, recycling the oligomers makes it possible to accelerate the rate of formation of the diester monomer (monomer of interest), in particular of BHET, while maintaining a rate of formation of BHETdeg that is identical to that in the case without recycling the oligomers;
a reduction in thermo-degradation phenomena by avoiding the presence of monomers and oligomers in the hottest zones of the process, in particular in conditioning step (a) and in the first reaction section of step (b). This effect contributes to improving the quality of the final product;
a reduction in the temperatures of the reaction sections which also makes it possible to improve the quality of the product and to improve the energy efficiency of the process.

According to the invention, said polyester feedstock may comprise at least coloured PET, opaque PET or mixtures thereof.

The conditioning step a) may implement at least one conditioning section (a1) for producing a fluid feedstock stream, and a mixing section (a2) for producing a mixed stream, where said mixed stream corresponds to the conditioned feedstock stream, said conditioning section (a1) being fed at least with said polyester feedstock and being carried out at a temperature between 150 and 300° C., said mixing section (a2) being fed at least with said fluid feedstock stream coming from the conditioning section and a diol stream, at least a portion of said diol stream being preferably composed of at least one fraction of the diol effluent from step c), said mixing section (a2) being operated at a temperature between 15° and 300° C., with a residence time between 0.5 second and 20 minutes.

The conditioning section (a1) can be operated in an extruder, said mixing section (a2) for the polyester feedstock of step a) possibly also being optionally implemented within said extruder.

The mixing section (a2) may implement at least one static or dynamic mixer.

In step a), the weight ratio of the diol stream introduced in step a) relative to the polyester feedstock may be between 0.03 and 6.00, preferably between 0.05 and 5.00, more preferably between 0.10 and 4.00, very preferably between 0.50 and 3.00.

Said at least one second reaction section can be operated at a temperature which is 5 to 50° C. lower than the temperature of said first reaction section.

The recycled oligomers effluent which feeds said at least one second reaction section may comprise the entirety of the first fraction originating from step e).

According to one particular embodiment, in step e), the second fraction may be divided into two parts, a first part being injected into one of the reaction sections of step b) downstream of the first reaction section, the second part being discharged from the process.

According to the invention, the depolymerization step may comprise two reaction sections. Alternatively, according to the invention, the depolymerization step may comprise three reaction sections, the second reaction section and the third reaction section each being fed with a part, varying between 0 and 100% by weight, of the recycled oligomers effluent, the sum of said parts constituting 100% by weight of said recycled oligomers effluent.

The depolymerization step may comprise at least one internal recirculation loop implementing at least the following operations: withdrawing a fraction of the reaction system from one of the reaction sections, filtering said fraction, and reinjecting said fraction into one of the reaction sections.

Separation step e) may be carried out so as to obtain a first fraction enriched in oligomers and a second fraction enriched in heavy impurities, the first fraction enriched in oligomers comprising a content of oligomers strictly greater than the content of oligomers of said heavy impurities effluent and the second fraction comprising a content of heavy impurities strictly greater than the content of heavy impurities of said heavy impurities effluent.

The first fraction obtained in step e) can be mixed with a diol stream, preferably with a fraction of the diol effluent obtained in step c) before being recycled to step b) and/or optionally to step a).

The diol separation step c) can be carried out in 1 to 5 successive gas-liquid separation sections each producing a gas effluent and a liquid effluent, the liquid effluent from the previous section feeding the subsequent section, the liquid effluent from the last gas-liquid separation section constituting the liquid monomer-rich effluent, and the collective gaseous effluents being recovered to constitute the diol effluent.

According to one improvement of the invention, the conditioning section can also be fed with a diol stream.

### LIST OF FIGURES

FIG. 1 represents a diagram of the process according to the invention. FIG. 1 is explained below.

FIG. 2 represents a particular diagram of the process according to the invention and illustrates the process according to the invention described in Example 1.

FIG. 3 represents a diagram of a process for depolymerization of a polyester not in accordance with the invention, comprising recycling to the conditioning step a), and illustrates the process described in Comparative Example 2.

FIG. 4 represents a diagram of a process for depolymerization of a polyester not in accordance with the invention, comprising recycling to the reactor A, and illustrates the process described in Comparative Example 3.

FIG. 5 represents a diagram of a process for depolymerization of a polyester not in accordance with the invention, and illustrates the process described in Comparative Example 4.

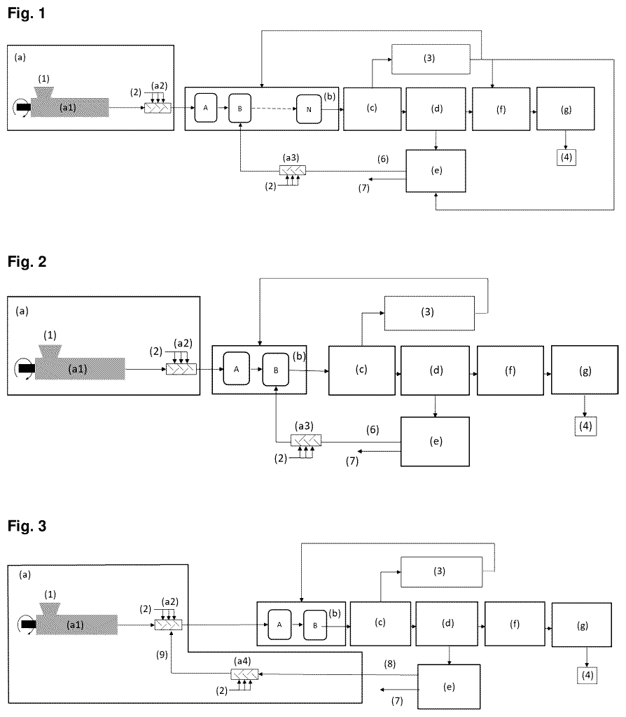

The references used in FIGS. 2 to 5 that are identical to the references used in FIG. 1 represent and designate the same elements.

### DESCRIPTION OF THE EMBODIMENTS

FIG. 1 illustrates a diagram of a process according to the invention without limiting its scope.

In the embodiment described with reference to FIG. 1, the process implements a step (a) of conditioning the feedstock (1) comprising PET. The conditioning step (a) uses a conditioning section (a1) (for example an extruder) to condition the feedstock (1) and obtain a fluid feedstock, at least one mixing section (a2) (for example a static or dynamic mixer, or an extruder portion) fed with the fluid feedstock, and a diol stream (2), which can advantageously be a fraction of the diol effluent (3) recovered in step (c). The mixed stream (or conditioned feedstock stream) obtained at the end of step (a) is introduced into the depolymerization step (b) which uses several reaction sections, for example: a first reaction section (A) and a second reaction section (B), and even other reaction sections. In FIG. 1, only the reaction sections (A), (B) and (N) are represented. Without departing from the scope of the invention, there may be other reaction sections between the reaction section (B) and the reaction section (N). The mixture passes successively into the reaction section (A) and then into the reaction section (B), optionally into a reaction section not shown, and then into the reaction section (N). Each of the reaction sections may be fed with diol (3) from step (c). The effluent obtained at the end of the depolymerization step (b) is introduced into the diol separation step (c), making it possible to recover a diol effluent (3) and an effluent rich in monomers.

The monomer-rich effluent is introduced into separation step (d), making it possible to obtain a pre-purified monomers effluent and to remove a heavy impurities effluent, comprising oligomers and heavy impurities, which is sent to separation step (e).

In separation step (e), the heavy impurities effluent is separated into two fractions, a first fraction (6) constituting the recycled oligomers effluent which feeds step (b) and a second fraction (7) which is discharged from the process according to the invention, in other words which is purged. According to the invention, at least 70% by weight of the first fraction constituting the recycled oligomers effluent which feeds step (b) is injected into the second reaction section (B).

Before being recycled to step (b), the recycled oligomers effluent can be mixed in a static or dynamic mixer (a3) with a diol stream (2), which can advantageously be a fraction of the diol effluent (3) recovered in step (c).

The pre-purified monomers effluent obtained at the end of step d) can be sent to a step (f) of decolourization by adsorption, then a crystallization step (g) to recover a decolourized purified diester monomer effluent (4).

The diol effluent (3) obtained in step (c) is advantageously recycled, in whole or in part, to step (b) in one or more of the reaction sections (A), (B) and (N) or an optional additional reaction section between section (B) and (N). In addition, a fraction of the diol effluent (3) obtained in step (c) can be recycled to one or more of the following steps: to step (f), to step (a), as a diol stream (2), for example in the static mixer (a2) and/or in step (e), being mixed with the heavy impurities effluent before separation and/or in the mixer (a3) to be mixed with the recycled oligomer effluent (6) before being sent to step (b).

According to the invention, polyethylene terephthalate or poly(ethylene terephthalate), also simply called PET, has an elementary repeating unit of formula:

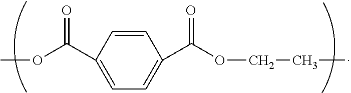

Conventionally, PET is obtained by polycondensation of terephthalic acid (PTA) or dimethyl terephthalate (DMT) with ethylene glycol. In the text hereinbelow, the expression “per mole of diester in said polyester feedstock” corresponds to the number of moles of —[O—CO—O—(C₆H₄)—CO—O—CH₂—CH₂]— unit, which is the diester unit obtained from the reaction of PTA and ethylene glycol, in the PET included in said polyester feedstock.

According to the invention, the term “monomer” or “diester monomer” advantageously designates the monomer of interest. Preferably, the monomer of interest is bis(2-hydroxyethyl) terephthalate (BHET) of chemical formula HOC₂H₄—CO₂—(C₆H₄)—CO₂-C₂H₄OH, in which —(C₆H₄)— represents an aromatic ring, and which is the diester unit obtained from the reaction of PTA and ethylene glycol, in the PET included in said polyester feedstock. Comonomers exist, defined by the occurrence of a single terephthalic unit, but different from BHET. The most predominant is 2-(2-hydroxyethoxy)ethyl 2-hydroxyethyl terephthalate (BHETdeg) with chemical formula HOC₂H₄—CO₂—(C₆H₄)—CO₂—C₂H₄O—C₂H₄OH and CAS #65133-69-9.

The term “oligomer” typically denotes a small-sized polymer, generally consisting of 2 to 20 elementary repeating units. According to the invention, the term “ester oligomer” or “diester oligomer” or alternatively “BHET oligomer” denotes a terephthalate ester oligomer comprising between 2 and 20, preferably between 2 and 5, elementary repeating units of formula —[O—CO—(C₆H₄)—CO—O—C₂H₄]—, with —(C₆H₄)— being an aromatic ring.

The term “heavy impurities” designates in particular pigments, polymers optionally present and which are other than the polyester, and polymerization catalysts.

According to the invention, the terms “diol” and “glycol” are used interchangeably and correspond to compounds comprising 2 hydroxyl groups —OH. The preferred diol is ethylene glycol, also called mono-ethylene glycol or MEG.

The diol or diol effluent streams used in the steps of the process of the invention, thus preferably comprise ethylene glycol (or MEG) in very predominant amounts, i.e. such that the MEG represents 95% by weight or more relative to the total weight of said diol or diol effluent stream.

The term “dye” defines a substance that is soluble in the polyester material and that is used to colour it. The dye may be of natural or synthetic origin.

According to the invention, the term “pigment”, more particularly colouring and/or opacifying pigment, defines a finely divided substance insoluble in particular in the polyester material. The pigments are in the form of solid particles, generally of between 0.1 and 10 μm and mostly of between 0.4 and 0.8 μm in size. They are often mineral in nature. The pigments generally used, notably for opacifying, are metal oxides, such as TiO₂, CoAl₂O₄ or Fe₂O₃, silicates, polysulfides and carbon black.

According to the present invention, the expressions “of between . . . and . . . ” and “between . . . and . . . ” mean that the limiting values of the interval are included in the range of values described. If this is not the case and the limiting values are not included in the range described, such precision will be provided by the present description.

In the sense of the invention, the various parameter ranges for a given step, such as the pressure ranges and the temperature ranges, can be used alone or in combination. For example, in the sense of the present invention, a range of preferred pressure values can be combined with a range of more preferred temperature values.

In the following, particular and/or preferred embodiments of the invention are described. They may be implemented separately or combined with each other without limitation of combinations where technically feasible.

According to the present invention, the pressures are absolute pressures. They are given in MPa (or MPa).

According to the invention, the times and durations are expressed in hours (h), minutes (min) and/or seconds (sec).

The terms “upstream” and “downstream” should be understood as a function of the general flow of the fluid(s) or stream(s) under consideration in the process.

### Feedstock

The process according to the invention is fed with a polyester feedstock comprising at least one polyester, that is to say a polymer whose main-chain repeating unit contains an ester function, and comprising polyethylene terephthalate (PET), preferably comprising at least coloured PET and/or opaque PET, or mixtures thereof.

Said polyester feedstock is advantageously a polyester feedstock to be recycled, originating from waste collection and sorting channels, in particular for plastic waste. Said polyester feedstock may come, for example, from the collection of bottles, trays, films, resins and/or fibres made of polyethylene terephthalate.

Advantageously, the polyester feedstock comprises at least 50% by weight, preferably at least 70% by weight and in a preferred manner at least 90% by weight of polyethylene terephthalate (PET).

Preferably, said polyester feedstock comprises at least one PET chosen from coloured, opaque, dark and multilayer PET, and mixtures thereof. In a very particular manner, said polyester feedstock comprises at least 10% by weight of opaque PET, very preferably at least 15% by weight of opaque PET, said opaque PET advantageously being opaque PET to be recycled, that is to say originating from collection and sorting channels.

Said polyester feedstock advantageously comprises from 0.1% to 10% by weight of pigments, advantageously from 0.1% to 5% by weight. It may also notably comprise from 0.05% to 1% of dyes, preferably from 0.05% to 0.2% by weight.

In the collection and sorting channels, the polyester resin waste is washed and ground before constituting the polyester feedstock of the process according to the invention. The polyester fibre waste is densified in the form of granules or popcorn so as to be able to be introduced mechanically into the process according to the invention.

The polyester feedstock may therefore be, in whole or in part, in the form of granules or flakes, the greatest length of which is less than 10 cm, preferably between 5 and 25 mm, or in the form of a micronized solid, that is to say in the form of particles preferably having a size of between 10 microns and 1 mm. The feedstock may also comprise “macroscopic” impurities, preferably less than 5% by weight, more preferably less than 3% by weight of “macroscopic” impurities, such as glass, metal, plastics other than polyester (for example PP, HDPE, etc.), wood, paper card, mineral elements. Said polyester feedstock may also be, in whole or in part, in the form of fibres, such as textile fibres, optionally pretreated to remove cotton fibres, polyamide fibres, or any other textile fibre other than polyester, or such as tyre fibres, optionally pretreated to remove, in particular, polyamide fibres or rubber or polybutadiene residues. Said polyester feedstock may further comprise polyester from the production scrap of the polyester material polymerization and/or transformation processes. The polyester feedstock may also comprise elements used as polymerization catalyst and as stabilizing agents in PET production processes, such as antimony, titanium, tin.

### Step a) of Conditioning

Said process according to the invention comprises a conditioning step a) which is fed with at least the polyester feedstock and optionally a diol stream in order to produce a conditioned feedstock stream. The conditioning step a) makes it possible to make the polyester feedstock at least partially liquid. Indeed, step a) makes it possible to bring the polyester feedstock to the operating conditions, temperature and pressure, of the depolymerization step b) and to render it at least partially liquid.

Preferably, said polyester conditioning step a) is carried out at a temperature between 15° and 300° C., preferably between 225 and 275° C. This temperature is sufficiently high to be close to or slightly higher than the melting temperature of the polyester, in particular PET, of the polyester feedstock, so that the latter is at least partially liquid, but is kept as low as possible to minimize degradation of the polyester. Preferably, the conditioning step a) is operated under an inert atmosphere in order to limit the introduction of oxygen into the system and the oxidation of the polyester feedstock.

Preferably, said conditioning step a) implements at least one conditioning section and one mixing section, said conditioning section being fed at least by said polyester feedstock and producing a fluid feedstock stream, said mixing section being fed at least with said fluid feedstock stream and at least one diol stream, said mixing section producing a mixed stream. In this preferred embodiment, the mixed stream corresponds to the conditioned feedstock stream which is recovered at the outlet of step a) and which advantageously feeds the depolymerization step b).

Said conditioning section of step a) makes it possible to heat and pressurize said polyester feedstock to the operating conditions of depolymerization step b). In the conditioning section, the polyester feedstock is gradually heated to a temperature close to or slightly higher than its melting temperature so as to become at least partially liquid. Advantageously, at least 70% by weight of the polyester feedstock, very advantageously at least 80% by weight, preferably at least 90% by weight, preferentially at least 95% by weight of the polyester feedstock is in liquid form on leaving the conditioning section of step a). The temperature at which the conditioning section of step a) is carried out is advantageously between 15° and 300° C., preferably between 225 and 275° C. Preferably, the conditioning section is operated under an inert atmosphere to limit the introduction of oxygen into the system and the oxidation of the polyester feedstock. The conditioning section may be operated with the addition of a diol stream to the polyester feedstock. In this case, the weight ratio of diol relative to the polyester feedstock in the conditioning section is between 0.001 and 0.1, preferably between 0.002 and 0.05, more preferably between 0.003 and 0.03.

According to one preferred embodiment of the invention, said conditioning section is an extrusion section which corresponds to a screw conveyor section. In other words, the conditioning section is operated in an extruder. The residence time in said extrusion section, defined as the volume of said section divided by the feedstock volume flow rate, is advantageously less than or equal to 5 h, preferably less than or equal to 1 h, more preferably less than or equal to 30 min, very preferably less than or equal to 10 min and preferably greater than or equal to 2 min. Advantageously, the extrusion section makes it possible to condition the polyester feedstock such that the stream of fluid feedstock is at a temperature between 15° and 300° C., preferably between 225 and 275° C., and at a pressure between atmospheric pressure (i.e. 0.1 MPa) and 20 MPa.

Said extrusion section is advantageously connected to a vacuum extraction system so as to remove impurities, such as dissolved gases, light organic compounds and/or moisture present in the feedstock. Said extrusion section may also advantageously comprise a filtration system for removing solid particles of a size greater than 40 μm, and preferably less than 2 cm, such as sand particles. The polyester feedstock is advantageously fed into the extruder by any method known to those skilled in the art, for example via a feed hopper, and is advantageously inertized to limit the introduction of oxygen into the system.

The mixing section is fed at least by said fluid feedstock stream coming from the conditioning section and by a diol stream. Said mixing section advantageously makes it possible to bring said polyester feedstock, previously treated in the conditioning section, into contact with a diol stream. This contacting has the effect of initiating the depolymerization reaction of the polyester feedstock, before introduction into the depolymerization step b). It also makes it possible to substantially reduce the viscosity of the feedstock, which facilitates its transportation, notably into the depolymerization step b). Very advantageously, said diol stream which feeds the mixing section is at least partly composed of at least one fraction of the diol effluent from step c) of the process.

In this preferred embodiment, said mixing section of step a) (also called mixing section for the polyester feedstock) is advantageously carried out at a temperature between 15° and 300° C., preferably between 225 and 275° C., at a residence time between 0.5 second and 20 minutes, preferably 1 second and 5 minutes, more preferably between 3 seconds and 3 minutes, and such that the weight ratio of the diol stream introduced in step a) (i.e. in the mixing section and optionally in the conditioning section) relative to the polyester feedstock is between 0.03 and 6.00, preferably between 0.05 and 5.00, more preferably between 0.10 and 4.00, very preferably between 0.50 and 3.00. The residence time is defined here as the ratio between the volume of liquid in said mixing section and the volume flow rate of diester feedstock in said mixing section.

In a first embodiment, said mixing section may implement at least one static or dynamic mixer, preferably between one and five successive static or dynamic mixers.

In a second very advantageous embodiment and when the conditioning section is operated in an extruder, the mixing section for the polyester feedstock can therefore be implemented within the extruder. In this case, it is a reactive extrusion phase.

The diol stream and the stream of diol, if used, can each be composed of a diol makeup external to the process according to the invention, a fraction of the diol effluent from step c), or mixtures thereof, preferably a fraction of the diol effluent from step c). Preferably, the diol stream and/or the stream of diol is/are advantageously heated prior to being fed in step a) in order to facilitate the heating of the polyester feedstock.

### Step b) of Depolymerization

The process according to the invention comprises a depolymerization step implementing a first reaction section and at least one second reaction section, said at least one second reaction section operating at a temperature strictly lower than said first reaction section, the first reaction section being fed with at least the conditioned feedstock stream and optionally with a first diol makeup, said at least one second reaction section being fed with the effluent from the first reaction section, a recycled oligomers effluent, and optionally with a second diol makeup. Said recycled oligomers effluent comprises at least 70% by weight of the first fraction separated in step e).

The depolymerization reaction corresponds to a depolymerization reaction by alcoholysis, preferably by glycolysis, that is to say preferably in the presence of diol.

Advantageously, the depolymerization step is operated such that the total amount of diol feeding said step b), corresponding to the sum of the amounts of diol introduced in step a) and in step b), is adjusted from 1 to 20 moles, preferably from 3 to 15 moles, more preferably from 5 to 10 moles of diol per mole of diester feeding said step b). In other words, the depolymerization step is advantageously operated such that the weight ratio between the total amount of diol introduced in step a) and step b) relative to the total amount of diester contained in the conditioned feedstock stream and optionally the fraction of the recycled oligomers effluent from step e) recycled to step b) is respectively between about 0.3 and 6.7, preferably between about 1.0 and 5.0, more preferably between 1.7 and 3.3.

Advantageously, said depolymerization step b) implements at least two reaction sections, for example between 2 and 4 reaction sections, preferably two or three reaction sections. The reaction sections operate in series, i.e. the effluent from one reaction section feeds a downstream reaction section. For example, the effluent from the first reaction section feeds the second reaction section, the effluent from the second reaction section feeds the third reaction section, and so on. Each reaction section can be used in any type of reactor known to those skilled in the art making it possible to carry out a depolymerization or transesterification reaction, preferably in a reactor stirred by a mechanical stirring system or/and by a recirculation loop or/and by fluidization. Said reactor may include a conical bottom for purging the impurities.

According to the invention, the reaction section(s) starting from the second reaction section is/are operated at a temperature strictly lower than the temperature of the first reaction section, preferably at a temperature lower by 5 to 50° C., preferably lower by 10 to 50° C., or even lower by 20 to 40° C., relative to the temperature of the first reaction section.

Said reaction sections are operated at a temperature of between 18° and 300° C., preferably between 19° and 300° C., more preferably between 200° C. and 280° C., in particular in the liquid phase.

The residence time in the depolymerization step, that is to say the cumulative residence time in the reaction sections used in the depolymerization step, is between 0.334 and 10 h, preferably between 0.5 and 8 h, and more preferably between 1 and 6 h. The residence time is defined as the ratio of the volume of liquid in said reaction sections to the volume flow rate of the stream leaving the last reaction section.

The operating pressure of said reaction section(s) of step b) is determined so as to maintain the reaction system in the liquid phase. This pressure is advantageously at least 0.1 MPa, preferentially at least 0.4 MPa, and preferably less than 5 MPa. The term “reaction system” means all of the constituents and phases present in said step b) obtained from the feeding of said step.

The diol is advantageously monoethylene glycol.

The first diol makeup which optionally feeds the first reaction section of step b) may be composed of a diol makeup external to the process according to the invention, a fraction of the diol effluent from step c), or mixtures thereof, and preferably of a fraction of the diol effluent from step c).

The second diol makeup which optionally feeds said at least one section reaction section of step b) may be composed of a diol makeup external to the process according to the invention, a fraction of the diol effluent from step c), or mixtures thereof, and preferably of a fraction of the diol effluent from step c).

The depolymerization reaction may be carried out in the presence or absence of a catalyst.

When the depolymerization reaction is carried out in the presence of a catalyst, the latter may be homogeneous or heterogeneous and chosen from esterification catalysts known to those skilled in the art, such as oxide complexes and salts of antimony, tin and titanium, alkoxides of metals of groups (I) and (IV) of the Periodic Table of the Elements, organic peroxides, and acid-base metal oxides.

A preferred heterogeneous catalyst advantageously comprises at least 50% by mass relative to the total mass of the catalyst, preferably at least 70% by mass, advantageously at least 80% by mass, very advantageously at least 90% by mass, and even more advantageously at least 95% by mass of a solid solution consisting of at least one spinel of formula ZₓAl₂O₍₃₊ₓ₎ in which x is between 0 (excluded limit) and 1, and Z is chosen from Co, Fe, Mg, Mn, Ti, Zn, and comprising not more than 50% by mass of alumina and oxide of the element Z. Said preferred heterogeneous catalyst advantageously contains at most 10% by mass of dopants chosen from silicon, phosphorus and boron, taken alone or as a mixture. For example, and in a non-limiting manner, said solid solution may consist of a mixture of ZnAl₂O₄ spinel and CoAl₂O₄ spinel, or else it may consist of a mixture of ZnAl₂O₄ spinel, MgAl₂O₄ spinel and FeAl₂O₄ spinel, or it may consist solely of ZnAl₂O₄ spinel.

Preferably, said depolymerization step is carried out without adding an external catalyst to the polyester feedstock.

Said depolymerization step may advantageously be carried out in the presence of a solid adsorbent in powder or shaped form, the function of which is to capture at least a portion of the coloured impurities, thus relieving the decolourization step f). Said solid adsorbent is advantageously an activated carbon.

The depolymerization reaction advantageously makes it possible to convert the polyester feedstock into ester monomers and oligomers, advantageously PET into at least one diester monomer and ester oligomers, in particular the bis(2-hydroxyethyl) terephthalate monomer (BHET) and BHET oligomers. The conversion of the polyester feedstock in said depolymerization step is greater than 50%, preferably greater than 70%, more preferably greater than 85%. The molar yield of diester monomer, in particular of BHET, is greater than 50%, preferably greater than 70%, more preferably greater than 85%. The molar yield of diester monomer, in particular of BHET, corresponds to the molar flow rate of diester monomer, in particular of BHET, at the outlet of said step b) over the number of moles of diester (that is to say of diester units) in the polyester feedstock feeding said step b).

An internal recirculation loop can advantageously be used in step b), that is to say the withdrawal of a fraction of the reaction system from one of the reaction sections, preferably the filtration of this fraction, and the reinjection of said fraction into one of the reaction sections. The reaction system corresponds to the fluid contained in each of the reaction sections.

According to one embodiment, the fraction can be reinjected into the same reaction section from which the fraction has been withdrawn. Alternatively, the fraction can be withdrawn from one of the reaction sections and said fraction can be reinjected into another reaction section different from that from which the withdrawal was carried out. The process according to the invention may comprise several withdrawal loops. For example, each of the reaction sections is equipped with a recirculation loop, each of the recirculation loops being able to carry out a withdrawal and a reinjection into the same reaction section. This (these) internal loop(s) make(s) it possible to eliminate the solid, “macroscopic”, impurities, possibly included in the reaction liquid.

Advantageously, the depolymerization step b) makes it possible to obtain a reaction effluent which is sent to a diol separation step c).

### Step c) of Separation of the Diol

The process according to the invention comprises a diol separation step c), fed at least with the effluent from step b), performed at a temperature of between 10° and 250° C., at a pressure below that of step b) and producing a diol effluent and an effluent rich in liquid monomers.

The main function of step c) is to recover all or part of the unreacted diol.

Step c) is performed at a lower pressure than that of step b) so as to vaporize a fraction of the effluent from step b) to give a gas effluent and a liquid effluent. Said liquid effluent constitutes the effluent rich in liquid monomers. The gaseous effluent, consisting of more than 50% by weight of diol, preferably more than 70% by weight, more preferably more than 90% by weight, constitutes a diol effluent.

Step c) is advantageously performed in a gas-liquid separation section or a succession of gas-liquid separation sections, advantageously from 1 to 5 successive gas-liquid separation sections, very advantageously from 3 to 5 successive gas-liquid separation sections. Each of the gas-liquid separation sections produces a liquid effluent and a gas effluent. The liquid effluent from the front section is fed to the subsequent section. The collective gaseous effluents are recovered to constitute the diol effluent. The liquid effluent obtained from the last gas/liquid separation section constitutes the effluent rich in liquid monomers.

Advantageously, one or even at least two gas-liquid separation sections can be used in a falling film evaporator or a scraped film evaporator or a short-path distillation.

Step c) is carried out in such a way that the temperature of the liquid effluents is maintained above the value below which the monomer and the polyester oligomers precipitate, and below a high value, depending on the molar ratio of diol to monomer, above which the monomer repolymerizes significantly. The temperature in step c) is between 10° and 250° C., preferably between 11° and 220° C., more preferably between 12° and 210° C. The operation in a succession of gas-liquid separations, advantageously in a succession of 2 to 5, preferably 3 to 5 successive separations, is particularly advantageous because it makes it possible to adjust in each separation the temperature of the liquid effluent that meets the abovementioned constraints.

A repolymerization inhibitor can advantageously be mixed with the liquid monomer-rich effluent before feeding said step c).

The pressure in step c) is below that in step b) and is advantageously adjusted to allow evaporation of the diol at a temperature while at the same time minimizing the repolymerization and enabling optimum energy integration. It is preferably between 0.00001 and 0.2 MPa, preferentially between 0.00004 and 0.15 MPa, with preference between 0.00004 and 0.1 MPa.

The separation section(s) are advantageously stirred via any method known to a person skilled in the art.

The diol effluent may contain other compounds such as dyes, acetaldehyde, dioxane, dioxolane, light alcohols, water, diamine monomers, urethane monomers, diols including diethylene glycol or cyclomethanediol. At least one fraction of the diol effluent is very advantageously recycled, in liquid form (that is to say after condensation), to step b) and/or optionally to step a), and optionally to step f), optionally mixed with an addition of diol external to the process according to the invention.

All or part of said diol effluent may be treated in a purification step (3) prior to its recycling, in liquid form (that is to say after condensation), advantageously to step b) and/or optionally to step a) and/or its use as a mixture in step e). This purification step may comprise, in a non-exhaustive manner, adsorption onto solid (for example onto activated carbon), in order to remove the dyes, and one or more distillations, in order to separate out the impurities, such as diethylene glycol, water and other alcohols.

The monomer-rich effluent obtained in step c) is sent to separation step d).

### Step d) of Separation of the Monomer

The process according to the invention comprises a step d) of separating out the monomer-rich effluent obtained from step c) producing a heavy impurities effluent and a pre-purified monomers effluent.

Said step d) is advantageously performed at a temperature of less than or equal to 250° C., preferably less than or equal to 230° C., and very preferably less than or equal to 215° C., and preferably greater than or equal to 110° C., and a pressure of less than or equal to 0.001 MPa, preferably less than or equal to 0.0005 MPa, preferably greater than or equal to 0.000001 MPa, with a liquid residence time of less than or equal to 10 min, preferably less than or equal to 5 min, preferably less than or equal to 3 min, and preferably greater than or equal to 0.1 second. The liquid residence time is defined as the ratio of the liquid volume in step d) to the volume flow rate of the liquid stream leaving step d).

The purpose of this separation step d) is to separate all or part of the monomer, in particular the BHET, which is vaporized, from the oligomers, which are not entirely converted, which remain liquid and therefore entrain oligomers corresponding to partially converted polyester polymer and also heavy impurities, particularly pigments, other polymers optionally present and polymerization catalysts, while minimizing the loss of monomers by repolymerization. Some oligomers may possibly be entrained with the monomer, in particular those small in size. These heavy impurities are found with the oligomers in the heavy impurities effluent.

Due to the possible presence of polymerization catalysts in the polyester feedstock, the separation must be performed with very short liquid residence times and at a temperature of less than or equal to 250° C., so as to limit any risk of re-polymerization of the monomer during this step. A separation by simple atmospheric distillation thus cannot be envisaged.

The separation step d) is advantageously performed in a falling-film or scraped-film evaporation system or by short-path falling film or scraped-film distillation. A very low operating pressure is necessary in order to be able to perform step d) at a temperature below 250° C., preferably below 230° C., while at the same time permitting vaporization of the monomer.

A polymerization inhibitor may advantageously be mixed with the liquid monomer-rich effluent before feeding said step d).

A fluxing agent may also advantageously be mixed with the liquid monomer-rich effluent before feeding said step d), so as to facilitate the removal of heavy impurities, particularly pigments, at the bottom of the evaporation or short-path distillation system. This fluxing agent must have a boiling point much higher than the monomer, in particular the BHET, under the operating conditions of step d). It may be polyethylene glycol, for example, or PET oligomers.

Said heavy impurities effluent in particular includes pigments, oligomers and unseparated monomer. Said heavy impurities effluent obtained in step d) is sent to the separation step e).

### Step e) of Separation of the Heavy Impurities Effluent

The process according to the invention comprises a step e) of separating said heavy impurities effluent from step d) so as to produce two fractions: a first fraction of which at least 70% by weight compose the recycled oligomers effluent which feeds step b), and a second fraction which is at least partly discharged from the process according to the invention.

The heavy impurities effluent obtained at the end of step d) comprises monomer, oligomers and heavy impurities, particularly pigments, unconverted polyester polymer, optionally other polymers and polymerization catalysts.

Preferably, the first fraction comprises at least 50% by weight, preferably at least 70%, more preferably at least 80% by weight, or even at least 90% by weight of the heavy impurities effluent obtained at the end of step d).

According to a first embodiment, step e) of separating said heavy impurities effluent consists of a simple division into two fractions, the two fractions having the same chemical composition.

According to a second embodiment, step e) of separating said heavy impurities effluent comprising the monomer, oligomers and heavy impurities is carried out so as to obtain a first fraction enriched in monomer and oligomers and a second fraction enriched in heavy impurities, meaning that the first fraction enriched in oligomers comprises a monomer and oligomer content strictly greater than the monomer and oligomer content of said heavy impurities effluent from step d) and that the second fraction comprises a heavy impurity content strictly greater than the heavy impurity content of said heavy impurities effluent. For example, the first fraction enriched in monomer and oligomers comprises a content of at least 10% by weight, preferably at least 50% by weight, more preferably at least 100% by weight of monomer and oligomers greater than the content of monomer and oligomers of said effluent of heavy impurities resulting from step d). For example, the second fraction enriched in heavy impurities comprises a content of at least 10% by weight, preferably at least 50% by weight, more preferably at least 100% by weight greater than the content of heavy impurities of said heavy impurities effluent from step d). In the second embodiment, the separation of said heavy impurities effluent can be carried out by filtration, by decantation, by MEG extraction, by centrifugation, etc. For example, said heavy impurities effluent from step d) may advantageously undergo at least one separation step, for example by filtration so as to reduce the amount of pigments and/or other solid impurities in the first fraction and recover a second fraction enriched in pigments and/or other solid impurities.

Preferably, the entirety of the first fraction obtained in step e) can be recycled to the depolymerization step b). More particularly, at least 70% by weight of this first fraction is injected into the or one of the reaction sections downstream of the first reaction section and the remainder, that is to say less than 30% by weight, of the first fraction is injected into another reaction section of step b), for example into the first reaction section. For example, in the embodiment of step b) with two reaction sections, the first reaction section is fed with less than 30% by weight, preferably less than 20% by weight, more preferably less than 10% by weight of the first fraction, and the second reaction section is fed with at least 70% by weight, preferably at least 80% by weight, more preferably at least 90% by weight of the first fraction.

For example, in the embodiment of step b) with three reaction sections, the first reaction section is fed with less than 30% by weight, preferably less than 20% by weight, more preferably less than 10% by weight of the first fraction, and the second reaction section and the third reaction section are each fed with a percentage of the first fraction varying between 0% by weight and 70% by weight, preferably between 0% by weight and 80% by weight, more preferably between 0% by weight and 90% by weight, the sum of percentages of the first fraction feeding the second and third sections being respectively at least 70% by weight, preferably at least 80% by weight, preferably at least 90% by weight of the first fraction. According to one particular embodiment of the invention, the entirety of the first fraction obtained in step e) is recycled by injection into the or one of the reaction sections downstream of the first reaction section. In other words, the first reaction section is not fed with the first fraction.

In order to facilitate separation, the heavy impurities effluent may be mixed with a diol effluent, for example with a portion of the diol effluent from step c), a diol makeup external to the process according to the invention, or mixtures thereof.

Before being recycled to step b), the first fraction can advantageously be mixed with a diol effluent, for example with a portion of the diol effluent from step c), a diol makeup external to the process according to the invention, or mixtures thereof. This mixing can be carried out in a static or dynamic mixer. Mixing the first fraction with a diol effluent facilitates recycling of ester oligomers, particularly BHET oligomers, since the mixing makes it possible on the one hand to fluidize the first fraction, which potentially concentrates solid particles such as pigments and polymeric compounds such as polyolefins, polyamides, polyurethanes present in the treated polyester feedstock and contributing to increasing the viscosity and fouling power of said residues, and thus to simplify the operability of their transport, and on the other hand to reduce the viscosity of said first fraction and therefore to promote its mixing with the polyester feedstock in the depolymerization step.

According to one embodiment, the entirety of the second fraction enriched in solid can advantageously be purged from the process, for example by being sent to an incineration system.

Alternatively, according to a second embodiment, a portion of the second fraction enriched in solid can be recycled to the depolymerization step, by injecting it into the or one of the reaction sections downstream of the first reaction section, the other remaining portion of the second fraction being purged from the process, for example by being sent to an incineration system.

In this second embodiment, the portion of the second fraction which is purged may represent at least 50% by weight, or even at least 70%, preferably at least 80% of the second fraction.

### Optional Step f) of Decolourizing

Advantageously, the process according to the invention may comprise a step of decolourizing the pre-purified monomers effluent from step d), operated at a temperature of between 7° and 180° C., preferably between 8° and 150° C., and more preferably between 9° and 120° C., and at a pressure of between 0.05 and 1.0 MPa, preferably between 0.05 and 0.8 MPa, and more preferably between 0.1 and 0.5 MPa in the presence of at least one adsorbent and producing a purified monomers effluent.

Said adsorbent may be any adsorbent known to those skilled in the art which is capable of taking up dyes, such as activated carbon or clays, advantageously an activated carbon.

The pre-purified monomers effluent may advantageously be mixed with a solvent. The pre-purified monomers effluent can be brought into contact with the solvent prior to being brought into contact with said adsorbent. Said solvent optionally introduced is, for example, a fraction of the diol effluent from step c) optionally previously treated in a purification step or, for example, with a diol makeup external to the process according to the invention or else, for example, with water.

### Optional Step g) of Crystallization

The purified monomers effluent obtained in optional step f) may optionally be introduced into a crystallization step g). When it is carried out, crystallization step g) advantageously implements at least one solid production section and at least one solid-liquid separation section.

Crystallization step g), when carried out, makes it possible to obtain a decolourized purified diester monomer effluent and discharges a used solvent effluent.

Advantageously, crystallization step g) implements one or more crystallization or precipitation operations and one or more solid-liquid separation operations. According to one particular embodiment, crystallization step g) uses a solid production section as described below, followed by a solid-liquid separation section as detailed below. According to another particular embodiment, crystallization step g) uses several solid production sections, preferably between two and five solid production sections, as described below, each of the solid production sections being followed by a solid-liquid separation section as detailed below.

The solid production section of step g) is advantageously fed with the purified monomers effluent from optional step f). Optionally, the solid production section may also be fed with a crystallization solvent, which is identical to or different from the solvent used in optional step f).

The crystallization solvent is advantageously chosen from water, monoalcohols, diols, ethers, aldehydes, esters, hydrocarbons and mixtures of at least two of these compounds belonging to the same chemical family or different chemical families. Preferably, said crystallization solvent is chosen from water, monoalcohols having between 1 and 12 carbon atoms, such as methanol or ethanol, and diols having between 1 and 12 carbon atoms. Very advantageously, said crystallization solvent is water, a monoalcohol having between 1 and 12 carbon atoms, such as methanol or ethanol, or a diol having between 1 and 12 carbon atoms, such as ethylene glycol. Preferably, the crystallization solvent is water, a diol having between 1 and 12 carbon atoms, preferably ethylene glycol, or mixtures thereof.

Preferably, when the decolourization and crystallization steps f) and g) are carried out and crystallization solvent is introduced in step g), the amount of crystallization solvent introduced into the solid production section is adjusted so that the purified monomers effluent which feeds step f) represents between 1 and 75% by weight, preferably between 5 and 45% by weight, more preferably between 15 and 35% by weight of the total weight of the mixture in said solid production section (i.e. the mixture comprising the purified monomers effluent, the solvent introduced in step f) and the crystallization solvent introduced in step g)).

Prior to being introduced into the solid production section, all or some of the crystallization solvent may be heated, preferably to the temperature at which the adsorption section is operated, or cooled and in particular brought to a temperature preferably of between 0 and 120° C., preferably between 5 and 100° C., and more preferably between 1° and 90° C.

Advantageously, the solid production section of optional step g) is operated at a temperature (that is to say such that the temperature of the effluent from said solid production section is) of between 0 and 100° C., preferably between 5 and 80° C., and more preferably between 1° and 70° C. More precisely, in the solid production section, the purified monomers effluent pretreated by adsorption, optionally mixed with the crystallization solvent, is cooled from the temperature at which the adsorption section is operated, that is to say from a temperature between 7° and 180° C., preferably between 80° C. and 150° C., more preferably between 9° and 120° C., to a temperature between 0 and 100° C., preferably between 5 and 80° C., and more preferably between 1° and 70° C.

The cooling may be implemented according to any method known to a person skilled in the art. For example, in particular in batch mode, the cooling of the temperature may be realized without regulation of the lowering of the temperature (i.e. without an imposed temperature ramp; thus only the initial and final temperatures are controlled) or according to at least one decreasing temperature ramp, in particular according to a decreasing temperature ramp of between 5 and 30° C./hour and more particularly between 8 and 15° C./hour, or else according to both modes chained together in succession, i.e. without control for one part of the cooling and according to a decreasing temperature ramp for another part of the cooling. According to another example, the cooling may simply be due to the introduction of the stream to be cooled, that is to say the monomer effluent pretreated by adsorption from adsorption step f) or the mixture comprising the monomer effluent pretreated by adsorption and the crystallization solvent, into a tank having a volume which is advantageously adapted to the flow rate of the stream to be cooled, maintained at a temperature of between 0 and 100° C., preferably between 5 and 80° C., and more preferably between 1° and 70° C.

The solid production section is advantageously operated at a pressure of between 0.00001 and 1.00 MPa, preferably between 0.0001 and 0.50 MPa, and with preference between 0.001 and 0.20 MPa. According to one particular embodiment of the invention, the solid production section is operated under vacuum, preferably at a pressure of between 0.0001 and 0.10 MPa, preferentially between 0.001 and 0.01 MPa. According to another particular embodiment, the solid production section is advantageously operated in a jacketed reactor, at a pressure between 0.01 and 1.00 MPa, preferably between 0.05 and 0.20 MPa, more preferably at atmospheric pressure, that is to say at 0.10 MPa.

Advantageously, the objective of the solid production section is at least in part to render solid, that is to say to crystallize or precipitate, the diester monomer, preferably BHET, present in the purified monomers effluent pretreated by adsorption and resulting from step f). Thus, the solid production section comprises, and preferably consists of, a precipitation or crystallization phase implemented by any precipitation or crystallization techniques known to a person skilled in the art. The solid production section is preferably a section for crystallization, for example by cooling or by concentration, which is implemented in any equipment known to a person skilled in the art, as defined, for example, in the journal Techniques de L'ingénieur, “Cristallisation industrielle—Aspects pratiques” [Industrial Crystallization—Practical Aspects], ref. J2788 V1, followed by a liquid-solid separation.

According to one preferred embodiment, water as crystallization solvent is mixed with the purified monomers effluent from step f) and the solid production section in step g) is operated under conditions such that the temperature of the effluent from said solid production section is between 5 and 50° C., preferably between 1° and 40° C.

According to another preferred embodiment, the crystallization solvent introduced and mixed with the purified monomers effluent from step f) is ethylene glycol and the solid production section is operated under conditions such that the temperature of the effluent from said solid production section is between 5 and 50° C., preferably between 1° and 40° C.

Advantageously, said solid production section, preferably operating by crystallization, comprises one or more crystallization operations, operating in series or in parallel, which is/are carried out batchwise or continuously, preferably continuously.

The solid production section makes it possible to obtain a heterogeneous effluent comprising a diester monomer solid phase and a liquid phase. The heterogeneous effluent is advantageously sent to the solid-liquid separation section.

In the solid-liquid separation section of optional step g), the diester monomer, preferably BHET, advantageously in solid form, in particular in crystal form, is separated from the liquid phase comprising all or part of the solvent introduced in step f) and the crystallization solvent optionally introduced into the solid production section. The solid-liquid separation section advantageously implements any means of solid-liquid separation known to a person skilled in the art, in particular at least one filtration, decantation and/or centrifugation system. The solid diester monomer thus separated constitutes the decolourized purified diester monomer effluent, the liquid phase constituting the used solvent effluent.

According to one particular embodiment, the decolourized purified diester monomer effluent, recovered at the end of optional step g) in solid form, preferably by filtration or centrifugation, can also advantageously undergo all or some of the following operations, carried out one or more times without a predefined chronological order: rinsing with a solvent identical to or different from the solvent feeding step f) or optionally the solid production section of step g); additional filtration or centrifugation; removal of the residual solvent by any method known to those skilled in the art, for example by evaporation drying; shaping, for example into powder or granules; and storage of the solid.

According to another embodiment, the decolourized purified diester monomer effluent is recovered, preferably by filtration or centrifugation, in the solid-liquid separation section and is then sent directly (that is to say without a solid storage phase) to a polymerization step known to those skilled in the art, optionally with, prior to the polymerization reaction, a rinsing with water or a diol effluent, for example an ethylene glycol effluent, preferably a rinsing with water, of the solid effluent of decolourized purified diester monomer, then the heating of the rinsed solid to be melted.

According to another embodiment, the decolourized purified diester monomer effluent is recovered, preferably by filtration or centrifugation, in the solid-liquid separation section, and the monomer is then dried by one of the means known to those skilled in the art before being stored or conveyed to a polymerization step known to those skilled in the art.

According to another embodiment, the decolourized purified diester monomer effluent is recovered, preferably by filtration or centrifugation, in the solid-liquid separation section and then the monomer is shaped after optionally having been dried by one of the means known to those skilled in the art (granulation, pelletization) before being stored or conveyed to a polymerization step known to those skilled in the art.

The decolourized purified monomer effluent resulting from optional step g) can advantageously feed a polymerization step known to those skilled in the art with a view to producing PET which is indistinguishable from virgin PET. The decolourized purified monomer effluent may advantageously be mixed with ethylene glycol, terephthalic acid or dimethyl terephthalate according to the polymerization step employed, prior to the polymerization reaction. The feed of the decolourized purified monomer effluent to a polymerization step makes it possible to reduce by an equivalent flow rate the feed of dimethyl terephthalate or terephthalic acid.

### EXAMPLES

Example 1, described with reference to FIG. 2, illustrates the invention without limiting its scope; Examples 2 to 4, described with reference to FIGS. 3 to 5 respectively, present examples not in accordance with the invention.

### Example 1 (According to the Invention)

The process of Example 1 is shown diagrammatically in FIG. 2.

The feedstock (1) composed of 100% PET is supplied continuously at a flow rate of 2500 kg/h, which corresponds to a recycling capacity of 20 KTA (kilotonnes per annum) of PET.

As illustrated in FIG. 2, the conditioning step (a) involves:
an extruder (a1) to condition the PET feedstock (1), by melting it,
a static mixer (a2) to premix the feedstock from the extruder with a stream (2) of ethylene glycol and obtain a mixed stream.

The mixed stream is introduced into the depolymerization step (b) which uses two reaction sections (A) and (B) arranged in series, each section being composed of a perfectly stirred reactor (reactor of the CSTR type, that is to say of Continuous Stirred Tank Reactor type). The working volumes of the reactors are: (A): 6 m³, (B): 42 m³. The reactor temperatures are: (A): 250° C., (B): 210° C.

The diol separation section (c) extracts a diol effluent comprising mainly ethylene glycol. The flow rate of the separated diol effluent is 9800 kg/h. The diol effluent is then purified, then mixed with a stream of fresh ethylene glycol with a flow rate corresponding to the consumption of the depolymerization, that is to say 700 kg/h in unit (3), and the whole is reinjected into reactor (A) for a flow rate of 7500 kg/h and into reactor (B) for a flow rate of 3000 kg/h. The stream of diols sent to the reactor (B) makes it possible to dilute the recycled oligomers effluent (6) coming from the separation section (e).

The separation section (d) for the effluent rich in monomers extracts the monomers from a heavy impurities effluent which comprises in particular the heavier oligomers, with a yield of 75%, thus generating a stream of pre-purified monomers having a flow rate of 2815 kg/h and an effluent of heavy impurities comprising heavier compounds (910 kg/h) and also a lost portion of monomers (940 kg/h). The effluent of heavy impurities (1850 kg/h), consisting of dimers and heavier compounds and also lost monomers and heavy impurities, is directed to the separation section (e). The stream of pre-purified monomers is discharged from (d) and introduced into the decolourization step (f) and then into the crystallization step (g) to produce a stream of purified and decolourized diester monomer (4) consisting of the monomer of interest BHET (2650 kg/h), the unwanted monomer BHETdeg (150 kg/h) and also compounds consisting in particular of monomer altered by thermal or thermo-oxidative degradation reactions (15 kg/h), which corresponds to a degree of purity of desired monomer of 94.1%.

The section for separating the heavy impurities effluent (e) is a simple non-selective division of the stream, which leads to two fractions: a first fraction (6), representing 80% of the feed stream of the separation section (e), which constitutes the recycled oligomers effluent, and a second fraction (7). The recycled oligomers effluent (6) (1480 kg/h) is entirely introduced into reactor (B), and the remainder (7) is purged (370 kg/h), that is to say discharged from the process.

Example 1 shows that directing the recycled oligomers effluent (6) to reactor (B) leads to the production of 15 kg/h of oxidized or thermally degraded species which are coloured and difficult to extract from the final monomer.

### Example 2 (Comparative: With Recycling to Conditioning Step a))

The process of Example 2 is shown diagrammatically in FIG. 3.

The feedstock (1) composed of 100% PET is supplied continuously at a flow rate of 2500 kg/h, which corresponds to a recycling capacity of 20 kilotonnes per year of PET.

As illustrated in FIG. 3, the conditioning step (a) involves:
an extruder (a1) to condition the PET feedstock (1), by melting it,
a static mixer (a3) to pre-mix the recycled oligomers effluent (8), comprising the oligomers from separation step (d), with a stream (2) of ethylene glycol, and obtain a residues mixture (9),
a static mixer (a2) for pre-mixing the feedstock from the extruder (a1) with a stream (2) of ethylene glycol and with the residues mixture (9) to obtain a mixed stream.

The mixed stream is introduced into the depolymerization step (b), which uses two reaction sections (A) and (B) arranged in cascade, each section being composed of a perfectly stirred reactor. The working volumes of the reactors are: (A): 6 m³, (B): 42 m³. The reactor temperatures are: (A): 250° C., (B): 210° C.

The diol separation section (c) extracts the diols perfectly, i.e. a flow rate of 9800 kg/h, from the terephthalics. The diols are then purified, mixed with a fresh stream having a flow rate corresponding to the consumption of the depolymerization, that is to say 700 kg/h in unit (3), and the major part is reinjected into reactor (A), the remainder into section (a3) and (a2), to accompany the recycled oligomers effluent (8).

The separation section (d) for the monomer-rich effluent extracts the monomers from the dimers and heavier oligomers with a yield of 75%, thus generating a stream of monomers pre-purified from the dimers and having a flow rate of 2827 kg/h and an effluent of heavy impurities comprising heavier compounds (870 kg/h) and also a lost portion of monomers (940 kg/h). The effluent consisting of dimers and heavier compounds and also lost monomers and heavy impurities, called heavy impurities effluent (1810 kg/h), is directed to the separation section (e). The purified monomers stream discharged from (d) is introduced into the decolourization step (f) and then into the crystallization step (g) to produce a purified and decolourized diester monomer stream (4) consisting of the monomer of interest BHET (2660 kg/h), the unwanted monomer BHETdeg (140 kg/h) and also monomer altered by thermal or thermo-oxidative degradation reactions (27 kg/h), which corresponds to a degree of purity of desired monomer of 94.1%.

The heavy impurities effluent separation section (e) partially extracts the heavy impurities from the terephthalics of the recycled residue stream with a yield of 20% to form a first fraction (8) which constitutes the recycled oligomers effluent and a second fraction (7). The recycled oligomers effluent (8) (1500 kg/h) is introduced into the static mixer (a3), and the remainder (7) is purged (375 kg/h), that is to say discharged from the process.

Example 2 shows that directing the recycled oligomers effluent to the static mixer (a4) and then (a2) of the conditioning step (a) increases the thermo-oxidative degradation phenomena by 80% relative to Example 1, by producing 27 kg/h of coloured species difficult to extract from the final monomer (whereas the process according to Example 1 produces only 15 kg/h of coloured species which are difficult to extract from the final monomer).

### Example 3 (Comparative: With Recycling to Reactor a)

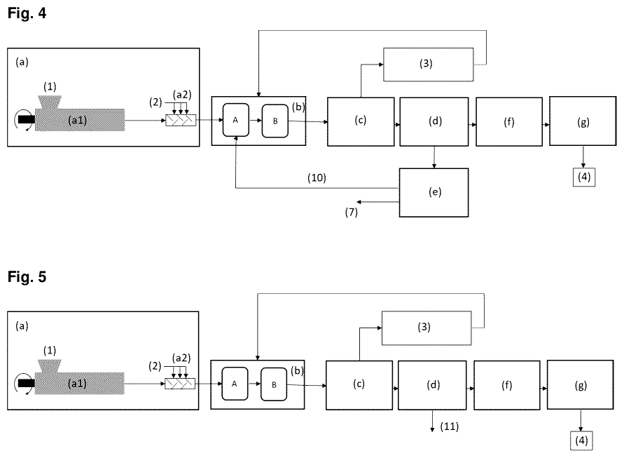

The process of Example 3 is shown diagrammatically in FIG. 4.

The feedstock (1) composed of 100% PET is supplied continuously at a flow rate of 2500 kg/h, which corresponds to a recycling capacity of 20 kilotonnes per year of PET.

As illustrated in FIG. 4, the conditioning step (a) involves:
an extruder (a1) to condition the PET feedstock (1), by melting it,
a static mixer (a2) to premix the feedstock from the extruder with a stream (2) of ethylene glycol and obtain a mixed stream.

The mixed stream is introduced into the depolymerization step (b), which uses two reaction sections (A) and (B) arranged in cascade, each section being composed of a perfectly stirred reactor. The working volumes of the reactors are: (A): 6 m³, (B): 42 m³. The reactor temperatures are: (A): 250° C., (B): 210° C.

The diol separation section (c) extracts the diols perfectly, i.e. a flow rate of 9800 kg/h, from the terephthalics. The diols are then purified, mixed with a fresh stream having a flow rate corresponding to the consumption of the depolymerization, that is to say 700 kg/h in unit (3), and the whole is reinjected into reactor (A) for a flow rate of 10 500 kg/h. The diol stream prepared in unit (3) is sent entirely to reactor (A) to accompany the mixed stream obtained in conditioning step (a) and the recycled oligomer effluent (6) coming from the separation section (e).

The separation section (d) for the monomer-rich effluent extracts the monomers from the dimers and heavier oligomers with a yield of 75%, thus generating a stream of monomers pre-purified from the dimers and having a flow rate of 2826 kg/h and an effluent of heavy impurities comprising heavier compounds (870 kg/h) and also a lost portion of monomers (940 kg/h). The effluent consisting of dimers and heavier compounds and also lost monomers and heavy impurities, called heavy impurities effluent (1810 kg/h), is directed to the separation section (e).

The purified monomers stream discharged from (d) is introduced into the decolourization step (f) and then into the crystallization step (g) to produce a purified and decolourized diester monomer stream (4) consisting of the monomer of interest BHET (2664 kg/h), the unwanted monomer BHETdeg (144 kg/h) and also monomer altered by thermal or thermo-oxidative degradation reactions (18 kg/h), which corresponds to a degree of purity of desired monomer of 94.3%.

The heavy impurities effluent separation section (e) partially extracts the heavy impurities from the terephthalics of the recycled residue stream with a yield of 20% to form a first fraction (10) which constitutes the recycled oligomers effluent and a second fraction (7). The recycled oligomers effluent (10) (1450 kg/h) is introduced into reactor (A), and the remainder (7) is purged (370 kg/h), that is to say discharged from the process.

Example 3 shows that directing the recycled oligomers effluent (10) to reactor (A) increases the thermo-oxidative degradation phenomena by 20% relative to Example 1, by producing 18 kg/h of coloured species difficult to extract from the final monomer (whereas the process according to Example 1 produces only 15 kg/h of coloured species which are difficult to extract from the final monomer).

### Example 4 (Comparative: Without Recycling of Heavy Impurities Effluent)

The process of Example 4 is shown diagrammatically in FIG. 5.

The feedstock (1) composed of 100% PET is supplied continuously at a flow rate of 2500 kg/h, which corresponds to a recycling capacity of 20 kilotonnes per year of PET.

As illustrated in FIG. 5, the conditioning step (a) involves:
an extruder (a1) to condition the PET feedstock (1), by melting it,
a static mixer (a2) to premix the feedstock from the extruder with a stream (2) of ethylene glycol and obtain a mixed stream.

The mixed stream is introduced into the depolymerization step (b), which uses two reaction sections (A) and (B) arranged in cascade, each section being composed of a perfectly stirred reactor. The working volumes of the reactors are: (A): 6 m³, (B): 42 m³. The reactor temperatures are: (A): 250° C., (B): 210° C.

The diol separation section (c) extracts the diols perfectly, i.e. a flow rate of 6900 kg/h, from the terephthalics. The diols are then purified, mixed with a fresh stream having a flow rate corresponding to the consumption of the depolymerization, that is to say 600 kg/h in unit (3), and the whole is reinjected into reactor (A) for a flow rate of 7500 kg/h.

The separation section (d) for the monomer-rich effluent extracts the monomers from the dimers and heavier oligomers with a yield of 75%, thus generating a stream of monomers pre-purified from the dimers and having a flow rate of 2380 kg/h and an effluent of heavy impurities comprising heavier compounds (710 kg/h) and also a lost portion of monomers (600 kg/h). Effluent (11) consisting of dimers and heavier compounds and also lost monomers and heavy impurities, called heavy impurities effluent (1310 kg/h), is discharged from the process, that is to say purged.

The purified monomers stream discharged from (d) is introduced into the decolourization step (f) and then into the crystallization step (g) to produce a purified and decolourized diester monomer stream (4) consisting of the monomer of interest BHET (2176 kg/h), the unwanted monomer BHETdeg (189 kg/h) and also monomer altered by thermal or thermo-oxidative degradation reactions (15 kg/h), which corresponds to a degree of purity of desired monomer of 91.4%.

Example 4 shows that the fact of not recycling part of the effluent of heavy impurities (10) leads to a purity of 91.4% being obtained, which is significantly lower than that of Example 1 (94.1%), accompanied by an overall monomer yield (71.1%) which is also significantly lower than that of Example 1 (84.5%). Moreover, the content of species resulting from thermal degradation reactions is similar to Example 1 (same flow rate, 15 kg/h, but represents a fraction of 0.6% by weight instead of 0.5% by weight according to Example 1 relative to the pre-purified monomer).

1. Process for depolymerizing a polyester feedstock comprising PET, said process comprising:
a) a conditioning step, fed at least by said polyester feedstock, to produce a conditioned feedstock stream;
b) a depolymerization step implementing a first reaction section (A) and at least one second reaction section (B), said at least one second reaction section (B) operating at a temperature strictly lower than the temperature of said first reaction section (A), the first reaction section (A) being fed at least by the conditioned feedstock stream and optionally by a first diol makeup, said at least one second reaction section (B) being fed by the effluent of the first reaction section, and by a recycled oligomers effluent and optionally by a second diol makeup, so that the total amount of diol feeding said step b) is adjusted to 1 to 20 moles of diol per mole of diester feeding said step b), said step b) being operated at a temperature of between 18° and 300° C. and with a residence time of between 0.334 and 10 h;
c) a step for separating the diol fed at least by the effluent of step b), operated at a temperature of between 10° and 250° C., at a pressure lower than that of step b) and producing a diol effluent and a liquid monomer-rich effluent;
d) a step for separating the liquid monomer-rich effluent from step c) into a heavy impurities effluent and a pre-purified monomers effluent, operated at a temperature of less than or equal to 250° C. and a pressure of less than or equal to 0.001 MPa with a liquid residence time of less than or equal to 10 min,
e) a step for separating said heavy impurities effluent into two fractions: a first fraction (6) of which at least 70% by weight compose the recycled oligomers effluent which feeds step b) and a second fraction (7) which is at least partially discharged from the process;
f) optionally, a step for decolourizing the pre-purified monomers effluent, operated at a temperature of between 10° and 250° C. and at a pressure of between 0.1 and 1.0 MPa in the presence of an adsorbent, and producing a purified monomers effluent,
g) optionally, a step for crystallizing the purified monomers effluent, employing at least one solid production section, operated at a temperature between 0 and 100° C. and at a pressure of between 0.00001 and 1.00 MPa, followed by a solid-liquid separation section, producing a decolourized and purified monomer effluent.

2. Process according to claim 1, in which said polyester feedstock comprises at least coloured PET, opaque PET or mixtures thereof.

3. Process according to claim 1, in which the conditioning step a) implements at least one conditioning section (a1) for producing a fluid feedstock stream, and a mixing section (a2) for producing a mixed stream, said mixed stream corresponding to the conditioned feedstock stream,
said conditioning section (a1) being fed at least by said polyester feedstock and being carried out at a temperature between 15° and 300° C.,
said mixing section (a2) being fed at least by said fluid feedstock stream coming from the conditioning section and a diol stream, at least a portion of said diol stream preferably being composed of at least a fraction of the diol effluent coming from step c), said mixing section (a2) being operated at a temperature between 15° and 300° C. with a residence time between 0.5 second and 20 minutes.

4. Process according to claim 3, in which the conditioning section (a1) of step a) is operated in an extruder, said section (a2) for mixing the polyester feedstock of step a) being optionally also implemented within said extruder.

5. Process according to claim 3, wherein the mixing section (a2) of step a) implements at least one static or dynamic mixer.

6. Process according to claim 3, in which, in step a), the weight ratio of the diol stream introduced in step a) with respect to the polyester feedstock is between 0.03 and 6.00, preferably between 0.05 and 5.00, more preferably between 0.10 and 4.00, very preferably between 0.50 and 3.00.

7. Process according to claim 3, in which said at least one second reaction section (B) is operated at a temperature lower by 5 to 50° C. with respect to the temperature of said first reaction section (A).

8. Process according to claim 3, in which the recycled oligomers effluent which feeds said at least one second reaction section (B) comprises the entirety of the first fraction originating from step e).

9. Process according to claim 3, in which, in step e), the second fraction (7) is divided into two parts, a first part being injected into one of the reaction sections of step b) downstream of the first reaction section, the second part being discharged from the process.

10. Process according to claim 3, in which the depolymerization step comprises two reaction sections.

11. Process according to claim 1, in which the depolymerization step comprises three reaction sections, the second reaction section and the third reaction section each being fed with a part, varying between 0 and 100% by weight, of the recycled oligomers effluent, the sum of said parts constituting 100% by weight of said recycled oligomers effluent.

12. Process according to claim 1, in which the depolymerization step comprises at least one internal recirculation loop implementing at least the following operations: withdrawing a fraction of the reaction system from one of the reaction sections, filtering said fraction, and reinjecting said fraction into one of the reaction sections.

13. Process according to claim 1, in which the separation step e) is carried out so as to obtain a first fraction (6) enriched in oligomers and a second fraction (7) enriched in heavy impurities, the first fraction enriched in oligomers comprising an oligomer content strictly greater than the oligomer content of said heavy impurities effluent and the second fraction comprising a heavy impurity content strictly greater than the heavy impurity content of said heavy impurities effluent.

14. Process according to claim 1, in which the first fraction (6) obtained in step e) is mixed (a3) with a diol stream, preferably with a fraction of the diol effluent obtained in step c) before being recycled to step b) and/or optionally to step a).

15. Process according to claim 1, in which the diol separation step c) is implemented in 1 to 5 successive gas-liquid separation sections each producing a gaseous effluent and a liquid effluent, the liquid effluent from the previous section feeding the subsequent section, the liquid effluent from the last gas-liquid separation section constituting the liquid monomer-rich effluent, and the collective gaseous effluents being recovered to constitute the diol effluent.

16. Process according to claim 3, in which the conditioning section is additionally fed with a diol stream.
